(19) United States
(12) Patent Application Publication
(10) Pub. No.: US 2010/0183692 A1
(43) Pub. Date: Jul. 22, 2010

(54) COMPOSITIONS

(76) Inventor: Andreas Natsch, Uetikon (CH)

Correspondence Address: CURATOLO SIDOTI CO., LPA, 24500 CENTER RIDGE ROAD, SUITE 280, CLEVELAND, OH, 44145, US

(21) Appl. No.: 12/665,490

(22) PCT Filed: Jun. 17, 2008
(86) PCT No.: PCT/CH08/00273

§ 371 Date: Dec. 18, 2009

(30) Foreign Application Priority Data
Jun. 22, 2007 (GB) 0712024.9

(51) Int. Cl. A61K 9/00; A61K 36/00; A61K 36/534; A61K 36/73; A61K 36/54; A61K 36/752; A61K 31/12; A61K 31/36; A61P 31/04

(52) U.S. Cl. 424/405; 424/725; 424/747; 424/765; 424/739; 424/736; 514/688; 514/464; 514/689

(57) ABSTRACT

Provided are essentially preservative-free personal care product compositions that comprise a combination of fragrance compounds that stabilises the personal care product against microorganisms. The combination comprises certain benzaldehyde or benzaldehyde derivative compounds combined with certain fragrant acids. The composition is free of classical preservatives including parabens or their derivatives. Further provided are methods of forming such compositions and products and the use of the combination of fragrance compounds in such compositions and products.

Provided are essentially preservative-free personal care product compositions and their use in personal care products applied to the human skin or scalp, and methods of making such products.

Preservatives are used in personal care products (products applied to the skin or scalp either to remain there or to be rinsed off) to preserve these products against microbial spoilage and to extend their shelf life. Antimicrobial compounds used for product preservation may fall into one or more of the following classes based on the effect they have on the microorganism, in particular bacteria and fungi. An antibacterial or antifungal may inhibit growth of the microorganisms or kill them or both. A bacteriostatic compound inhibits growth of bacteria, while a bactericide kills bacteria (reduces their number). Similarly, a fungistatic compound inhibits the growth of fungi (molds and yeast), while a fungicide kills fungi (reduces their number). A sporicide kills spores of fungi or bacteria. Spores, especially endospores, are formed by some bacteria to survive during periods of deprivation and are significantly more difficult to kill. Fungi form spores for reproduction and these spores are significantly more difficult to kill than the vegetative form of the fungi. Many antimicrobial compounds are therefore not effective against fungal spores.

A broad band preservative effect including a bactericidal and fungicidal activity was previously only attained in personal care products by addition of certain preservatives, in particular formaldehyde, formaldehyde donors, halogenated compounds, compounds belonging to the class of parabens and a variety of specific fungicides. Formaldehyde donors include in particular diazolidinyl urea (CAS 78491-02-8), imidazolidinyl urea (CAS 39236-46-9), and DMDM Hydantoin (CAS 6440-58-0).

Halogenated compounds include in particular 2,4-dichlorobenzyl-alcohol (CAS 1777-82-8), Chloroxylenol (also known as 4-chloro-3,5-dimethyl-phenol, CAS 88-04-0), Bronopol (also known as 2-bromo-2-nitropropane-1,3-diol, CAS 52-51-7), iodopropynyl butyl carbamate (CAS 55406-53-6).

Paraben compounds include in particular Methyl-paraben (CAS 99-76-3), Ethyl-paraben (CAS 120-47-8), Propyl-paraben (CAS 94-13-3), Butyl-paraben (CAS 94-26-8), Isopropyl-paraben (CAS 4191-73-5), and Benzyl-paraben (CAS 94-18-8).

Other preservatives include Quaternium-15 (CAS 51229-78-8), methyl-chloroisothiazolinone (CAS 26172-55-4), and methylisothiazolinone (CAS 2682-20-4).

There are concerns that some of these preservatives may constitute health hazards, for example, iodopropynyl butyl carbamate, formaldehyde and formaldehyde donors, methyl-chloroisothiazolinone (CAS 26172-55-4), and methylisothiazolinone are considered highly allergenic/sensitizing.

Accordingly there is an interest in removing preservatives from personal care products applied to human skin or scalp while maintaining a good broad band stabilisation of the product against microbial spoilage including a sporicidal effect.

Applicant surprisingly identified a combination of fragrance compounds that enables to provide essentially preservative-free personal care product compositions that display a stabilizing effect, in particular a broad-band antimicrobial action including a sporicidal effect. Due to their well known characteristics and their primary use as fragrance, the compounds of the discovered combination are not considered preservatives in the industry and accordingly do not have to be labelled as such.

The stabilizing effect is reached by the following combination of fragrance compounds:

a) benzaldehyde and benzaldehyde-derivatives according to formula I as defined herein below and
b) fragrant acids selected from the group consisting of cinnamic acid and anisic acid according to formula II as defined herein-below.

Some of these benzaldehyde and benzaldehyde derivative compounds have previously been shown to have a fungistatic effect against various food spoilage molds and yeasts. The antifungal activity of a given antifungal against a given fungal species varies with the food product in which it is used, possibly due to the concentration of lipids or proteins. Fitzgerald et al. report vanillin and various derivatives to have antifungal (fungistatic) activity against a variety of food molds including various Aspergillus species (A. oryzae, A sojae), Penicillium species, and yeast strains when tested in yeast extract peptone dextrose broth. The efficacy against various fungal strains varies. Fungicidal or sporicidal activities were not tested. (J. Agric. Food Chem. 2005, 53, 1769-1775).

Similarly, heliotropin is known to be active as a fungistatic compound in vaporous form when applied to fungi on tobacco leaves, and to have an antifungal and antibacterial effect against some fungi and bacteria in aqueous culture media.

While many substituted benzaldehydes and benzylalcohols are known to have a germistatic activity against some microorganisms, the germicidal effect, in particular the bactericidal and fungicidal effect, is generally considered to be low, especially when the pH is within the range commonly used in personal care products which is pH 5 to pH 9. While some compounds are known to be more active under extremely acidic or alkaline conditions, this effect does not extend to the pH range used in personal care products.

That compounds that are fungistatic in certain food stuffs can provide a fungicidal and sporicidal effect in personal care products that often contain lipids and proteins or a high concentration of detergents was completely surprising and could not have been predicted. An activity or lack of activity of a given test compound in water is not indicative of an activity in a personal care product, for example a cosmetic cream.

### SUMMARY

In a first aspect, there is provided a personal care product composition comprising

a) at least one benzaldehyde or benzaldehyde-derivative compound of formula I,

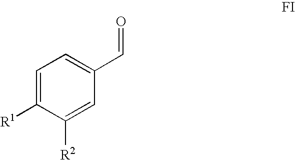

wherein R¹ is a residue selected from the group consisting of H, CH₂, CH₃, OH, and OCH₃,
wherein R² is a residue selected from the group consisting of H, OH, OCH₃, and OCH₂O, and
wherein if R² is OCH₂O, then R¹ is CH₂ and forms a bond with R², selected from the group consisting of Benzaldehyde, 4-methylbenzaldehyde, Heliotropine, Vanillin, 4-hydroxybenzaldehyde, 3-hydroxybenzaldehyde, 4-methoxybenzaldehyde, 3-methoxybenzaldehyde, and 3-hydroxy-4-methoxybenzaldehyde (isovanillin); and
b) at least one fragrant acid according to formula II

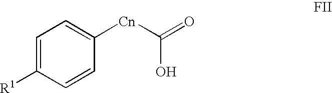

wherein R¹ is a residue selected from the group consisting of H and OCH₃,
wherein n is selected from 0 or 2, and
wherein if R¹ is H then n is 2, and the C atoms of the resulting C₂ situated between aromatic ring and COOH form a double bond, and
wherein if R¹ is OCH₃ then n is 0;
selected from the group consisting of cinnamic acid and anisic acid;
wherein the at least one benzaldehyde or benzaldehyde-derivative compound a) is present in a total concentration of 0.05 to 0.5% (w/w);
wherein the at least one fragrant acid b) is present in a total concentration of 0.05 to 0.5% (w/w);
and a cosmetically-acceptable base,
with the proviso that the personal care product composition is essentially free of a preservative selected from the group consisting of Formaldehyde paraformaldehyde, Biphenyl-2-ol (o-phenylphenol) and its salts, Pyrithione zinc, Inorganic sulphites and hydrogen-sulphites, Sodium iodate, Chlorobutanol, 4-Hydroxybenzoic acid and its salts and esters, 3-Acetyl-6-methylpyran-2,4(3H)-dione (Dehydracetic acid) and its salts, Formic acid and its sodium salt, 3,3′-Dibromo-4,4′-hexamethylenedioxydibenzamidine (Dibromohexamidine) and its salts (including isethionate), Thiomersal, Phenylmercuric salts (including borate), Hexetidine, 5-Bromo-5-nitro-1,3-dioxane, Bronopol, 2,4-Dichlorobenzyl alcohol, Triclocarban, 4-Chloro-m-cresol, 4-Chloro-3,5-xylenol, 3,3′-Bis(1-hydroxymethyl-2,5-dioxoimidazolidin-4-yl)-1,1′-methylenediurea (‘Imidazolidinyl urea’), Hexamethylenetetramine (methenamine), Methenamine 3-chloroallylochloride, 1-(4-Chlorophenoxy)-1-(imidazol-1-yl)-3,3-dimethylbutan-2-one, 1,3-Bis(hydroxymethyl)-5,5-dimethylimidazolidine-2,4-dione, 1-Hydroxy-4-methyl-6(2,4,4-trimethylpentyl) 2-pyridon and its monoethanolamine salt, 1,2-Dibromo-2,4-dicyanobutane (methyldibromoglutaronitrile), 6,6-Dibromo-4,4-dichloro-2,2′-methylenediphenol (Bromochlorophen), Mixture of 5-Chloro-2-methyl-isothiazol-3(2H)-one and 2-methylisothiazol-3(2H)-one with magnesium chloride and magnesium nitrate, 2-Benzyl-4-chlorophenol (chlorophene), 2-Chloroacetamide, Chlorhexidine and its digluconate, diacetate and dihydrochloride, Alkyl (C12-C22) trimethyl ammonium, bromide and chloride, 4,4-dimethyl-1,3-oxizalidine, N-(Hydroxymethyl)-N-(dihydroxymethyl-1,3-dioxo-2,5-imidazolidinyl-4)-N′-(hydroxymethyl)urea, 1,6-Di(4-amidinophenoxy)-n-hexane (Hexamidine) and its salts (including isethionate and p-hydroxybenzoate), Glutaraldehyde (Pentane-1,5-dial), 5-Ethyl-3,7-dioxa-1-azabicyclo[3.3.0]octane, 3-(p-chlorophenoxy)-propane-1,2 diol (chlorphenesin), Silver chloride deposited on titanium dioxide, Benzethonium Chloride, Benzalkonium chloride, bromide and saccharinate, Benzylhemiformal, 3-Iodo-2-propynylbutylcarbamate, and Methylisothiazolinone.

In another aspect, there is provided a personal care product composition as described herein, wherein the at least one benzaldehyde or benzaldehyde-derivative compound a) is present in a concentration of 0.075 to 0.3% (w/w), optionally 0.1 to 0.2%, (w/w), and wherein the at least one fragrant acid b) is present in a concentration of 0.075 to 0.3% (w/w), optionally 0.1 to 0.2% (w/w).

In another aspect, there is provided a personal care product composition as described herein, further comprising at least one aromatic alcohol or derivative thereof selected from the group consisting of phenylethyl alcohol, phenyl propyl alcohol, benzyl formiate, and phenethyl formiate.

In another aspect, there is provided a personal care product composition as described herein, further comprising an antimicrobial fragrance ingredient selected from the group consisting of Geranium oil, Peppermint oil, Rose oil, Cinnamon leaf oil, Fucus oil, Clove bud oil, Clove leaf oil, Palmarosa oil, Citrus oil, Terpene fraction of citrus oil, Orange oil, Terpene fraction of orange oil, Geraniol, Cuminic alcohol, Perilla alcohol, Citronellol, Eugenol, Cinnamic alcohol, Nerol, Menthol, Borneol, Octan-1-ol, Nonan-1-ol, Decan-1-ol, Dec-9-en-1-ol. Limonene, 2,6-dimethyl-7-octen-2-ol, 3-methyl-5-phenyl-pentanol, 2-methyl-5-phenyl-pentanol, undec-10-en-1-ol, 4-(1-methylethyl)-cyclohexanol, 4-(1,1-dimethylethyl)-cyclohexanol, 2,2-dimethyl-3-(3-methyl phenyl)-propanol, 4-(1-methylethyl)cyclohexyl-methanol, (E)-2-(3,3-dimethylbicyclo[2.2.1]hept-2-ylidene)-ethanol, 3,7-dimethyl-7-octen-1-ol, 2-methoxy-4-propyl-phenol, 3-(4-methyl-3-cyclohexenyl)-butanol, Dihydroterpineol, 2,6-dimethylheptan-2-ol, 3-(4-methoxyphenyl)-2-methyl-Propanal, Octahydro-5-methoxy-4,7-Methano-1H-indene-2-carboxaldehyde, alpha-methyl-1,3-Benzodioxole-5-propanal, 3-[4-(1,1-dimethylethyl)phenyl]propanal, 8-(1-methylethyl)-1-oxaspiro[4.5]-decan-2-one, Tetrahydro-6-(2Z)-2-pentenyl-2H-Pyran-2-one, 5-(3Z)-3-hexenyldihydro-2(3H)-Furanone, 5-hexyldihydro-5-methyl-2(3H)-Furanone, 4-methyl-5-pentyl-dihydro-2(3H)-Furan-2-one, 6-hexyltetrahydro-2H-pyran-2-one, 5-hexyl-Furan-2(3H)-one, 6-heptyltetrahydro-2H-Pyran-2-one, 5-octyl-furan-2-one, Dihydro-5-pentyl-2(3H)-Furanone, 5-heptyldihydro-(2(3H)-Furanone), and 6-hexyltetrahydro-2H-Pyran-2-one.

In another aspect, there is provided a personal care product composition as described herein, further comprising at least one aromatic alcohol or derivative thereof as described herein, and at least one antimicrobial fragrance ingredient as described herein.

In another aspect, there is provided a composition as described herein, selected from compositions for personal care products applied to and left on the skin or scalp including but not limited to creams, salves, lotions, and ointments for hand, face or body, perfumes, eau de Cologne, eau de toilet, deodorants, antiperspirants, and products applied but rinsed off including but not limited to soaps, liquid soaps, shower gels, and shampoos.

In another aspect, there is provided a personal care product comprising the composition as described herein, selected from an application form selected from stick, roll-on, spray, pump-spray, aerosol, soap bar, powder, solution, gel, cream, balm and lotion.

In another aspect, there is provided a personal care product or composition therefor as described herein, wherein the personal care product composition comprises lipids.

In another aspect, there is provided a personal care product or composition therefor as described herein, wherein the composition is an emulsion.

In another aspect, there is provided a personal care product or a personal care product composition as described herein, wherein the pH is 5 to 9.

In another aspect, there is provided a personal care product composition as described herein, wherein the benzaldehyde or benzaldehyde-derivative compound a) and the fragrant acid b) as described herein, the aromatic alcohols or derivatives thereof as described herein, and the antimicrobial fragrance ingredients as described herein that are present in the composition exclusively are compounds or ingredients that occur in nature.

In another aspect, there is provided a personal care product composition as described herein, wherein all ingredients of the composition consists exclusively of compounds or ingredients that occur in nature.

In another aspect, there is provided a personal care product composition as described herein, wherein the compounds or ingredients that occur in nature have been extracted or purified from a natural source including but not limited to a botanical source, or that have been formed by natural processes including but not limited to fermentation.

In another aspect, there is provided the use of at least one benzaldehyde or benzaldehyde-derivative compound a) as described herein, and at least one fragrant acid b) as described herein, for the preparation of a preserved personal care product composition, or a preserved personal care product.

In another aspect, there is provided a method of forming a stabilized personal care product which is sufficiently bactericidal to have a reduction factor for Pseudomonas aeruginosa and Staphylococcus aureus of at least 1000 per 7 days, and which is sufficiently sporicidal to have a reduction factor of at least 100 per 7 days for Aspergillus niger,

by admixing an effective amount of at least one benzaldehyde or benzaldehyde-derivative compound a) and an effective amount of at least one fragrant acid b) as described herein, to a personal care product base, wherein optionally said compounds a) and b) may be added in form of a pre-prepared fragrance composition comprising said compounds a) and b), forming a personal care product composition as described herein.

In another aspect, there is provided a method as described herein, wherein the total concentration of the at least one benzaldehyde or benzaldehyde-derivative compound a) in the personal care product composition is from 0.05% to 1% (w/w), optionally from 0.3 to 0.7%, and the total concentration of the at least one fragrant acid b) in the personal care product composition is from 0.05% to 1% (w/w), optionally 0.3 to 0.7%.

In another aspect, there is provided a method as described herein wherein the personal care product composition formed further comprises at least one aromatic alcohol or derivative thereof selected from the group consisting of phenylethyl alcohol, phenyl propyl alcohol, benzyl formiate, and phenethyl formiate.

In another aspect, there is provided a method as described herein, wherein the personal care product composition formed further comprises at least one antimicrobial fragrance ingredient as described herein.

In another aspect, there is provided a method as described herein, wherein the personal care product composition formed further comprises at least one aromatic alcohol or derivative thereof selected from the group consisting of phenylethyl alcohol, phenyl propyl alcohol, benzyl formiate, and phenethyl formiate; and at least one antimicrobial fragrance ingredient as described herein.

In another aspect, there is provided a method as described herein, wherein the personal care product composition is or is brought into the form of a personal care product including sticks, roll-ons, sprays, pump-sprays, aerosols, soap bars, powders, solutions, gels, creams, balms and lotions.

### DETAILED DESCRIPTION

The personal care products described herein are essentially preservative-free, meaning that they may contain only traces of preservatives as herein defined, whose concentration is substantially below their effective antimicrobial concentration, for example, at least 5 times or 10 times less. Such traces can be due to, for example, impurities of one or more ingredients of a personal care product or preserved ingredients/excipients used in low concentrations in personal care products.

The term “preservative” as used herein refers to a preservative or combination of preservatives listed herein below. Preservatives are substances which may be added to personal care products for the primary purpose of inhibiting the development of micro-organisms in such products. ‘Salts’ of preservatives is taken to mean: salts of the cations sodium, potassium, calcium, magnesium, ammonium and ethanolamines; salts of the anions chloride, bromide, sulphate, acetate. ‘Esters’ of preservatives is taken to mean: esters of methyl, ethyl, propyl, isopropyl, butyl, isobutyl, phenyl.

### Preservatives:

Formaldehyde paraformaldehyde
Biphenyl-2-ol (o-phenylphenol) and its salts
Pyrithione zinc (INN)
Inorganic sulphites and hydrogen-sulphites
Sodium iodate
Chlorobutanol (INN)
4-Hydroxybenzoic acid and its salts and esters
3-Acetyl-6-methylpyran-2,4(3H)-dione (Dehydracetic acid) and its salts
Formic acid and its sodium salt
3,3′-Dibromo-4,4′-hexamethylenedioxydibenzamidine (Dibromohexamidine) and its salts (including isethionate)
Thiomersal (INN)
Phenylmercuric salts (including borate)
Hexetidine (INN)
5-Bromo-5-nitro-1,3-dioxane
Bronopol (INN)
2,4-Dichlorobenzyl alcohol
Triclocarban (INN)
4-Chloro-m-cresol
4-Chloro-3,5-xylenol
3,3′-Bis(1-hydroxymethyl-2,5-dioxoimidazolidin-4-yl)-1,1′-methylenediurea (‘Imidazolidinyl urea’)
Hexamethylenetetramine (methenamine) (INN)
Methenamine 3-chloroallylochloride (INNM)
1-(4-Chlorophenoxy)-1-(imidazol-1-yl)-3,3-dimethylbutan-2-one
1,3-Bis(hydroxymethyl)-5,5-dimethylimidazolidine-2,4-dione
1-Hydroxy-4-methyl-6(2,4,4-trimethylpentyl) 2-pyridon and its monoethanolamine salt
1,2-Dibromo-2,4-dicyanobutane (methyldibromoglutaronitrile)
6,6-Dibromo-4,4-dichloro-2,2′-methylenediphenol (Bromochlorophen)
Mixture of 5-Chloro-2-methyl-isothiazol-3(2H)-one and 2-methylisothiazol-3(2H)-one with magnesium chloride and magnesium nitrate
2-Benzyl-4-chlorophenol (chlorophene)
2-Chloroacetamide
Chlorhexidine (INN) and its digluconate, diacetate and dihydrochloride
Alkyl (C12-C22) trimethyl ammonium, bromide and chloride
4,4-dimethyl-1,3-oxizalidine
N-(Hydroxymethyl)-N-(dihydroxymethyl-1,3-dioxo-2,5-imidazolidinyl-4)-N′-(hydroxymethyl)urea
1,6-Di(4-amidinophenoxy)-n-hexane (Hexamidine) and its salts (including isethionate and p-hydroxybenzoate)
Glutaraldehyde (Pentane-1,5-dial)
5-Ethyl-3,7-dioxa-1-azabicyclo[3.3.0]octane
3-(p-chlorophenoxy)-propane-1,2 diol (chlorphenesin)
Silver chloride deposited on titanium dioxide
Benzethonium Chloride (INCI)
Benzalkonium chloride, bromide and saccharinate
Benzylhemiformal
3-Iodo-2-propynylbutylcarbamate
Methylisothiazolinone (INCI)

The chemical structures of compounds a), benzaldehyde and certain benzaldehyde-derivatives, are shown below.

| | |
|---|---|
| Benzaldehyde | 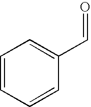 |
| 4-methylbenzaldehyde | 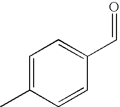 |
| Heliotropine | 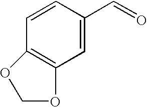 |
| Vanillin | 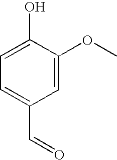 |
| 4-hydroxybenzaldehyde | 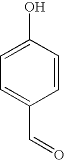 |
| 3-hydroxybenzaldehyde | 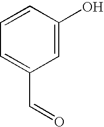 |
| 4-methoxybenzaldehyde | 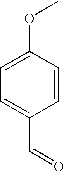 |
| 3-methoxybenzaldehyde | 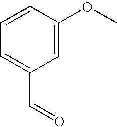 |
| 3-hydroxy-4-methoxybenzaldehyde (isovanillin) | 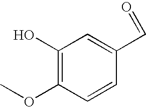 |

These benzaldehyde and benzaldehyde derivative compounds for use as described herein are commercially available.

Compounds b) are fragrant acids selected from cinnamic acid and anisic acid. Their chemical structures are shown below.

| | |
|---|---|
| cinnamic acid | 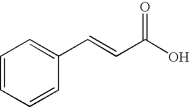 |
| anisic acid | 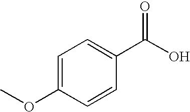 |

### Optional Ingredients

Optional ingredients, including optional ingredients to further improve the stabilization against microbial spoilage may be present in personal care products as herein described. In particular, the personal care product may contain one or more aromatic alcohol or derivative thereof as optional ingredient, in particular phenylethyl alcohol, phenyl propyl alcohol, benzyl formiate, and phenethyl formiate. These compounds are commonly used in personal care products as fragrance. Their structural formulae are shown below.

| Aromatic alcohol or derivative | structural formula |
|---|---|
| 2-Phenylethyl alcohol | 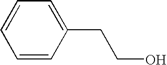 |
| 3-Phenyl propyl alcohol | 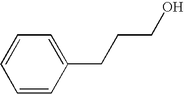 |
| Benzyl formiate | 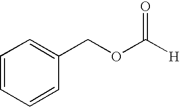 |
| Phenethyl Formiate | 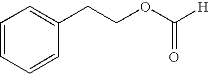 |

Further optional ingredients to further improve the stabilization of the products against microbial spoilage may be antimicrobial (bactericidal and/or fungicidal) fragrance ingredients or compounds.

Examples for the above-mentioned fragrance compounds are given below.

### Antimicrobial Fragrant Natural Oils Include but are not Limited to:

Geranium oil, Peppermint oil, Rose oil, Cinnamon leaf oil, Fucus oil, Clove bud oil, Clove leaf oil, Palmarosa oil, Citrus Oil, Terpene-fraction from citrus oil, Orange oil, Terpene-fraction from orange oil

### Antimicrobial Fragrance Compounds Found in Natural Essential Oils Include but are not Limited to:

Geraniol, Cuminic alcohol, Perilla alcohol, Citronellol, Eugenol, Cinnamic alcohol, Nerol, Menthol, Borneol, Octan-1-ol, Nonan-1-ol, Decan-1-ol, Dec-9-en-1-ol, and Limonene.

### Synthetic Antimicrobial Fragrance Alcohols Include but are not Limited to:

2,6-dimethyl-7-octen-2-ol (Dihydromyrcenol™)
3-methyl-5-phenyl-pentanol (Phenoxanol™),
2-methyl-5-phenyl-pentanol (Rosaphen™)
undec-10-en-1-ol (ALC C11 Undecylenique™),
4-(1-methylethyl)-cyclohexanol (Folrosia™)
4-(1,1-dimethylethyl)-cyclohexanol (P T Butyl Cyclohexanol™)
2,2-dimethyl-3-(3-methyl phenyl)-propanol (Majantol™),
4-(1-methylethyl)cyclohexyl-methanol (Mayol™),
(E)-2-(3,3-dimethylbicyclo[2.2.1]hept-2-ylidene)-ethanol (Patchomint™),
3,7-dimethyl-7-octen-1-ol (Rhodinol™),
2-methoxy-4-propyl-phenol (Dihydroeugenol™)
3-(4-methyl-3-cyclohexenyl)-butanol (Cyclomethyleneitronellol™)
Dihydroterpineol, and
2,6-dimethylheptan-2-ol (Dimetol™).

### Synthetic Antimicrobial Fragrance Aldehydes Include but are not Limited to:

3-(4-methoxyphenyl)-2-methyl-Propanal, (Fennaldehyde™)
Octahydro-5-methoxy-4,7-Methano-1H-indene-2-carboxaldehyde, (Scentenal™)
alpha-methyl-1,3-Benzodioxole-5-propanal, (Tropional™)
3-[4-(1,1-dimethylethyl)phenyl]propanal (Bourgeonal™)

### Synthetic Antimicrobial Fragrance Lactones Include but are not Limited to:

8-(1-methylethyl)-1-oxaspiro[4.5]-decan-2-one (Laitone™),
Tetrahydro-6-(2Z)-2-pentenyl-2H-Pyran-2-one, (Lactone Jasmin Delta™)
5-(3Z)-3-hexenyldihydro-2(3H)-Furanone, (Lactone Jasmin Gamma™)
5-hexyldihydro-5-methyl-2(3H)-Furanone, (Methyl Decalactone Gamma™), and
4-methyl-5-pentyl-dihydro-2(3H)-Furan-2-one, (Tuberate Methyl Pur™)

### Antimicrobial Fragrance Lactones Found in Nature Include but are not Limited to:

6-hexyltetrahydro-2H-pyran-2-one (Decalactone Delta™)
5-hexyl-Furan-2(3H)-one (Decalactone Gamma™)
6-heptyltetrahydro-2H-Pyran-2-one, (Dodecalactone Delta™)
5-octyl-furan-2-one, (Dodecalactone Gamma™)
Dihydro-5-pentyl-2(3H)-Furanone, (Prunolide™)
5-heptyldihydro-(2(3H)-Furanone), (Gamma-Undecalactone™)
6-hexyltetrahydro-2H-Pyran-2-one, (Delta-Undecalactone™)

The antimicrobial fragrance compounds may be synthesized or extracted from natural sources including but not limited to botanical sources, for examples fragrant natural oils derived from plants or part of plants, including but not limited to Geranium oil, Peppermint oil, Rose oil, Cinnamon leaf oil, Fucus oil, Clove bud oil, Clove leaf oil, Palmarosa oil, Citrus oil (for example: terpene fraction), and Orange oil (for example: terpene fraction).

Bases for personal care products are well known in the art and the resulting personal care product will usually have a pH of pH5 to pH9 (for example, slightly acidic for products applied to and left on the skin, slightly alkaline for soap products). It is also possible to employ an existing preservative-free personal care product composition and simply add a) and b) in the concentrations hereinabove defined and mix thoroughly.

The exact concentration of compounds under a) and b) that is employed in a composition will depend upon the nature of the product, the stabilisation effect against microbial spoilage (in particular the bactericidal, fungicidal and sporicidal activity), and the length of this effect to be achieved.

A useful concentration for the compound a) is, for example, without limitation, 0.05 to 0.5%, 0.075 to 0.3%, and 0.1 to 0.2% (w/w).

A useful concentration for the compound b) is, for example, 0.05 to 0.5%, 0.075 to 0.3%, and 0.1 to 0.2% (w/w).

If combined in the given concentrations, compounds a) and b) generally provide a sufficient bactericidal, fungicidal and sporicidal activity in a wide range of personal care product compositions.

In particular, a sufficient bactericidal activity is attained when the reduction factor is 1000 per 7 days. A sufficient sporicidal activity is attained when the reduction factor is 100 per 7 days. A sufficient sporicidal activity is strongly indicative of a sufficient fungicidal activity.

The reduction factor is determined by growing a suitable test organism (Aspergillus niger for fungi, Pseudomonas aeruginosa for gram-negative bacteria and Staphylococcus aureus for gram-positive bacteria) on a suitable culture medium on agar plates, harvesting and adding to a personal care product composition in a density of 3×10⁵ organism/ml and counting the surviving organisms in the probe and a negative control at defined time interval, commonly 7 days. The count of the negative control is divided by the count of the probe and thereby the reduction factor is determined (compare example 1).

b)-compounds of particular interest are 4-hydroxybenzaldehyde and 3-hydroxybenzaldehyde, for their surprisingly good activity.

The addition of hydroxy groups to benzaldehyde and derivatives was previously shown not to provide a fungicidal effect on A. niger.

Fitzgerald et al (who looked at fungistatic effects only, and only of certain food-relevant fungi excluding A. niger), found that the removal of hydroxy groups from 4-hydroxybenzaldehyde resulted in a slight improvement of fungistatic activity against certain food molds, and the only position beneficial for antifungal (fungistatic) activity was the 2-OH position within the benzene ring of benzaldehyde (J. Agric. Food Chem. 2005, 53, 1769-1775).

Personal care product compositions are used to form a personal care product in an appropriate application form and packaging, as is well-known in the art.

Personal care products and compositions to form them as described herein are used for the purpose of cleansing, conditioning, grooming, beautifying, promoting attractiveness, or otherwise enhancing or altering the appearance of the human body and are applied to the human skin or scalp.

These include products applied to and left on the skin or scalp, for example creams, salves, lotions, and ointments for hand, face or body, perfumes, eau de Cologne, eau de toilet, deodorants, antiperspirants, and products applied but rinsed off such as soaps, liquid soaps, shower gels, shampoos.

These products can, for example, take various forms of application, for example sticks, roll-ons, sprays, pump-sprays, aerosols, soap bars, powders, solutions, gels, creams, balms and lotions.

Many personal care products will be formulated as an emulsion or other lipid-containing products and these form a particular aspect of the embodiments described-herein. Lipids are often included for example into washing formulations including liquid soaps or washing lotions to provide an oil replenishing effect. The a) and b) compounds as hereinabove defined allow the formulation of stabilized emulsions or formulations comprising lipids and/or detergents where the activity (the bactericidal, fungicidal and in particular the sporicidal effect) is not lost due to the presence of the lipid base and/or detergents or surfactants.

Depending on the nature of the personal care product, personal care product compositions as described herein may also be combined with art-recognised quantities of other excipients commonly employed in these products; useful selections may be found in <<CTFA Cosmetic Ingredient Handbook>>, J. M. Nikitakis (ed.), 1st ed., The Cosmetic, Toiletry and Fragrance Association, Inc., Washington, 1988, which is hereby incorporated by reference.

In general, excipients may, for example, include colorants, fragrances, solvents, surfactants, colorants, opacifiers, buffers, antioxidants, vitamins, emulsifiers, UV absorbers, silicones and the like. All products can also be buffered to the desired pH using commonly-available excipients in a known manner.

There now follows a series of non-limiting examples that serve to illustrate the invention.

While the personal care product compositions, products, and related methods have been described above in connection with certain illustrative embodiments, it is to be understood that other similar embodiments may be used or modifications and additions may be made to the described embodiments for performing the same function. Further, all embodiments disclosed are not necessarily in the alternative, as various embodiments may be combined to provide the desired characteristics. Variations can be made by one having ordinary skill in the art without departing from the spirit and scope of the disclosure. Therefore, the compositions, products and methods should not be limited to any single embodiment, but rather construed in breadth and scope in accordance with the recitation of the attached claims.

### EXAMPLES

Unless otherwise indicated, all concentrations given are % w/w.

### Example 1

### Sporicidal Effect of Test Compounds in Water

Aspergillus niger ATCC 16404 spores are added to water to obtain a density of 3×10⁵ spores 1 ml. In order to prepare the spores, the test strain is grown for 5 days on potato dextrose agar at room temperature. The spores are harvested with a solution containing 0.1% Tween 80, peptone 0.1% and NaCl 0.85% and the spore concentration is adjusted to the density indicated above.

Test compounds are dissolved in dipropyleneglycol to a concentration of 20%.

These stock solutions are added to 10 ml aliquots of the spore suspension to obtain a final concentration of the test compounds of 0.1%. The sporicidal effect is shown by a reduction of spore counts after 7 days.

The reduction factor is determined as follows. Aliquots of the above prepared suspension of microorganisms (here: spore suspension prepared as described above) are plated on a suitable agar medium (see above) and the developing colonies are counted both for samples with test compound and for a negative probe (water). The count of the negative control is divided by the count of the test compound and thereby the reduction factor is determined. A negative control (water) accordingly has a reduction factor of 1 (no effect on the microorganism).

TABLE 1
Sporicidal effect of test compounds in water

0.1% test compound

| | | Reduction |
|---|---|---|
| Test compound | Spores/ml | factor |
| Negative control (water) | 2.5 × 10⁵ | 1 |
| 4-hydroxybenzaldehyde | >1.2 × 10⁵ | <2 |
| 4-methoxy-benzaldehyde | 4.68 × 10⁴ | 5 |

No significant reduction of spore counts is achieved with 4-hydroxybenzaldehyde and 4-methoxy-benzaldehyde.

### Example 2

### Sporicidal Effect of a) and b) Compounds in a Cosmetic Cream

A preservative-free cosmetic cream for application to the human skin is used (Cremor basalis, Fagron GmbH, Barsbüttel, Germany).

Test samples of cream contain different amounts of compounds a) and b) added to an aliquot of 10 g of the cream in 50 ml tubes to give a concentration (w/w) of 0.125-0.25% as shown in the table below. After addition of the compounds a) and b), the cream is thoroughly mixed to achieve a homogeneous distribution.

After 1-3 days of equilibration of the cream (storage at room temperature to achieve a homogenous partitioning of compounds between oil and water phase), to each sample 100 μl of a spore suspension of Aspergillus niger ATCC 16404 containing 3×10⁷ spores/ml (prepared as described in example 1) is added. After regular test intervals (7 days), samples of 1 g cream are removed and added to 20 ml of a neutralizer solution containing 0.2% lecithin, 2% Tween 80 and 0.5% NaCl. These dilutions are vigorously shaken for 10 min until the cream is dissolved, and then aliquots of this solution are spread plated on potato dextrose agar containing 0.2% Tween 80. After a sufficient time for colonies to grow (about 48 h), the number of colony forming units (and therefore surviving spores in the cream) are counted.

The table below shows the reduction factor after 7 days of A. niger spores of anisic acid or cinnamic acid (in form of their sodium salts) when combined with 4-hydroxybenzaldehyde (4-HBA) in a skin cream.

TABLE 1
Sporicidal effect of cosmetic cream samples with anisic acid and
cinnamic acid alone or in combination with 4-HBA.

| | Concentration | Concentration | |
|---|---|---|---|
| | Fragrant Acid | 4-HBA | Reduction factor |
| Fragrant Acid | [% w/w] | [% w/w] | (*) |
| none | 0 | 0.125 | 5.5 |
| Anisic acid | 0.250 | 0 | 12.5 |
| Anisic acid | 0.250 | 0.125 | >1000 |
| Anisic acid | 0.125 | 0 | 1.4 |
| Anisic acid | 0.125 | 0.125 | >1000 |
| Cinnamic acid | 0.250 | 0 | 1.1 |
| Cinnamic acid | 0.250 | 0.125 | >1000 |
| Cinnamic acid | 0.125 | 0 | 1.0 |
| Cinnamic acid | 0.125 | 0.125 | 225.8 |

Surprisingly, though 4-hydroxybenzaldehyde shows no significant sporicidal effect in water (compare example 1), this and several other test compounds show an excellent activity when combined with cinnamic acid or anisic acid in cream at 0.125% concentration (complete killing of spores within 7 days).

The results show that the sporicidal effect on Aspergillus niger spores is only achieved when combining anisic acid or cinnamic acid with 4-HBA. Neither one of the fragrant acids alone achieves the sporicidal effect necessary for stabilisation of personal care products (reduction factors of maximally up to 12.5). Nor does 4-HBA achieve a significant sporicidal effect in cosmetic cream, at 0.125% concentration the reduction factor is 5.5.

Similar results are achieved when employing, instead of 4-HBA, another a) compound selected from Benzaldehyde, 4-methylbenzaldehyde, Heliotropine, Vanillin, 4-hydroxybenzaldehyde, 3-hydroxybenzaldehyde, 4-methoxybenzaldehyde, 3-methoxybenzaldehyde, and 3-hydroxy-4-methoxybenzaldehyde (isovanillin).

### Example 3

### Bactericidal Effect of a) and b) Compounds in a Cosmetic Cream

Test samples of the same cream used in Example 2 contain different amounts of compounds a) and b) added to an aliquot of 10 g of the cream in 50 ml tubes to give a concentration (w/w) of 0.125-0.25% as shown in the table below. After addition of the compounds a) and b), the cream is thoroughly mixed to achieve a homogeneous distribution.

As test organisms Staphylococcus aureus (DSMZ 799) and Pseudomonas aeruginosa (ATCC 15442) are used. The strains are grown overnight in Mueller-Hinton broth and adjusted to a cell density of 1×10⁸ cfu (colony forming units) per ml.

The two bacterial strains are mixed in a ratio of 1:1 and 100 μl of this mixed inoculum is added to 10 ml aliquots of the cosmetic cream.

The resulting mixtures are incubated at room temperature and at the regular intervals samples are removed, suspended in neutralizer solution and diluted as described above (example 2).

Aliquots of these suspended and diluted samples are plated on tryptic soy agar supplemented with 0.5% Tween 80 and then incubated for 24 h at 37° C. Surviving bacteria are counted.

The table below shows the reduction factor of bacterial counts after 24 h for anisic acid or cinnamic acid (in form of their sodium salts) when combined with 4-hydroxybenzaldehyde (4-HBA) in a skin cream.

TABLE 2
bactericidal effect of cosmetic cream samples with anisic acid and
cinnamic acid alone or in combination with 4-HBA.

| | Concentration | Concentration | |
|---|---|---|---|
| | Fragrant Acid | 4-HBA | Reduction factor |
| Fragrant Acid | [% w/w] | [% w/w] | (*) |
| none | | 0.125 | 10 |
| Anisic acid | 0.250 | 0 | 5 |
| Anisic acid | 0.250 | 0.125 | >1000 |
| Anisic acid | 0.125 | 0 | 1 |
| Anisic acid | 0.125 | 0.125 | >1000 |
| Cinnamic acid | 0.250 | 0 | 10 |
| Cinnamic acid | 0.250 | 0.125 | >1000 |
| Cinnamic acid | 0.125 | 0 | 2.0 |
| Cinnamic acid | 0.125 | 0.125 | >1000 |

Similar results are achieved when employing, instead of 4-HBA, another a) compound selected from Benzaldehyde, 4-methylbenzaldehyde, Heliotropine, Vanillin, 4-hydroxybenzaldehyde, 3-hydroxybenzaldehyde, 4-methoxybenzaldehyde, 3-methoxybenzaldehyde, and 3-hydroxy-4-methoxybenzaldehyde (isovanillin).

These results show that bactericidal effects to prevent the product from microbial spoilage can be achieved by combining a compound selected from the compound a) with a compound selected from the compounds b).

1. A personal care product composition comprising
a) at least one benzaldehyde or benzaldehyde-derivative compound of formula I, 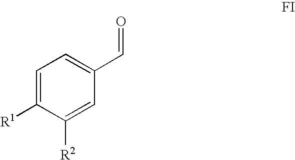
wherein R¹ is a residue selected from the group consisting of H, CH₂, CH₃, OH, and OCH₃,
wherein R² is a residue selected from the group consisting of H, OH, OCH₃, and OCH₂O, and
wherein when R² is OCH₂O, then R¹ is CH₂ and forms a bond with R², selected from the group consisting of Benzaldehyde, 4-methylbenzaldehyde, Heliotropine, Vanillin, 4-hydroxybenzaldehyde, 3-hydroxybenzaldehyde, 4-methoxybenzaldehyde, 3-methoxybenzaldehyde, and 3-hydroxy-4-methoxybenzaldehyde; and
b) at least one fragrant acid according to formula II 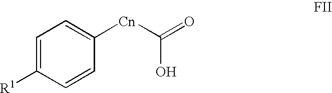
wherein R¹ is a residue selected from the group consisting of H and OCH₃,
wherein n is selected from 0 or 2, and
wherein if R¹ is H then n is 2, and the C atoms of the resulting C₂ situated between aromatic ring and COOH form a double bond, and
wherein if R¹ is OCH₃ then n is 0;
selected from the group consisting of cinnamic acid and anisic acid;
wherein the at least one benzaldehyde or benzaldehyde-derivative compound a) is present in a total concentration of 0.05 to 0.5% (w/w);
wherein the at least one fragrant acid b) is present in a total concentration of 0.05 to 0.5% (w/w);
and a cosmetically-acceptable base,
with the proviso that the personal care product composition is essentially free of a preservative selected from the group consisting of Formaldehyde, Biphenyl-2-ol, Biphenyl-2-ol salts, Pyrithione zinc, Inorganic sulphites, hydrogen-sulphites, Sodium iodate, Chlorobutanol, 4-Hydroxybenzoic acid, 4-Hydroxybenzoic acid salts, 4-Hydroxybenzoic acid esters, 3-Acetyl-6-methylpyran-2,4(3H)-dione, 3-Acetyl-6-methylpyran-2,4(3H)-dione salts, Formic acid, sodium salt of Formic acid, 3,3′-Dibromo-4,4′-hexamethylenedioxydibenzamidine, Dibromohexamidine, 3,3′-Dibromo-4,4′-hexamethylenedioxydibenzamidine salts, (3,3′-Dibromo-4,4′-hexamethylene-dioxydibenzamidine isethionate salt, Thiomersal, Phenylmercuric salts, Phenylmercuric borate, Hexetidine, 5-Bromo-5-nitro-1,3-dioxane, Bronopol, 2,4-Dichlorobenzyl alcohol, Triclocarban, 4-Chloro-m-cresol, 4-Chloro-3,5-xylenol, 3,3′-Bis(1-hydroxymethyl-2,5-dioxoimidazolidin-4-yl)-1,1′-methylenediurea, Imidazolidinyl urea, Hexamethylenetetramine, Methenamine 3-chloroallylochloride, 1-(4-Chlorophenoxy)-1-(imidazol-1-yl)-3,3-dimethylbutan-2-one, 1,3-Bis(hydroxymethyl)-5,5-dimethylimidazolidine-2,4-dione, 1-Hydroxy-4-methyl-6(2,4,4-trimethylpentyl) 2-pyridon, 1-Hydroxy-4-methyl-6(2,4,4-trimethylpentyl) 2-pyridon monoethanolamine salt, 1,2-Dibromo-2,4-dicyanobutane, 6,6-Dibromo-4,4-dichloro-2,2′-methylenediphenol, Mixture of 5-Chloro-2-methyl-isothiazol-3(2H)-one and 2-methylisothiazol-3(2H)-one with magnesium chloride and magnesium nitrate, 2-Benzyl-4-chlorophenol, 2-Chloroacetamide, Chlorhexidine, Chlorhexidine digluconate, Chlorhexidine diacetate, Chlorhexidine dihydrochloride, Alkyl (C12-C22) trimethyl ammonium, Alkyl (C12-C22) trimethyl bromide, Alkyl (C12-022) trimethyl chloride, 4,4-dimethyl-1,3-oxizalidine, N-(Hydroxymethyl)-N-(dihydroxymethyl-1,3-dioxo-2,5-imidazolidinyl-4)-N′-(hydroxymethyl)urea, 1,6-Di(4-amidinophenoxy)-n-hexane, Hexamidine, salts of 1,6-Di(4-amidinophenoxy)-n-hexane, 1,6-Di(4-amidinophenoxy)-n-hexane isethionate salt, 1,6-Di(4-amidinophenoxy)-n-hexane p-hydroxybenzoate salt, Glutaraldehyde, 5 Ethyl-3,7-dioxa-1-azabicyclo[3.3.0]octane, 3-(p-chlorophenoxy)-propane-1,2 diol, Silver chloride deposited on titanium dioxide, Benzethonium Chloride, Benzalkonium chloride, Benzalkonium bromide, Benzalkonium saccharinate, Benzylhemiformal, 3-Iodo-2-propynylbutylcarbamate, and Methylisothiazolinone.

2. The personal care product composition according to claim 1 wherein the at least one benzaldehyde or benzaldehyde-derivative compound a) is present in a concentration of 0.075 to 0.3% (w/w), optionally 0.1 to 0.2% (w/w), and wherein the at least one fragrant acid b) is present in a concentration of 0.075 to 0.3% (w/w), optionally 0.1 to 0.2% (w/w).

3. The personal care product composition according to claim 1 further comprising at least one aromatic alcohol or derivative thereof selected from the group consisting of phenylethyl alcohol, phenyl propyl alcohol, benzyl formiate, and phenethyl formiate.

4. The personal care product composition according to claim 1 further comprising an antimicrobial fragrance ingredient selected from the group consisting of Geranium oil, Peppermint oil, Rose oil, Cinnamon leaf oil, Fucus oil, Clove bud oil, Clove leaf oil, Palmarosa oil, Citrus oil, Terpene fraction of citrus oil. Orange oil, Terpene fraction of orange oil, Geraniol, Cuminic alcohol, Perilla alcohol, Citronellol, Eugenol, Cinnamic alcohol, Nerol, Menthol, Borneol, Octan-1-ol, Nonan-1-ol, Decan-1-ol, Dec-9-en-1-ol, Limonene, 2,6-dimethyl-7-octen-2-ol, 3-methyl-5-phenyl-pentanol, 2-methyl-5-phenyl-pentanol, undec-10-en-1-ol, 4-(1-methylethyl)-cyclohexanol, 4-(1,1-dimethylethyl)-cyclohexanol, 2,2-dimethyl-3-(3-methyl phenyl)-propanol, 4-(1-methylethyl)cyclohexyl-methanol, (E)-2-(3,3-dimethylbicyclo[2.2.1]hept-2-ylidene)-ethanol, 3,7-dimethyl-7-octen-1-ol, 2-methoxy-4-propyl-phenol, 3-(4-methyl-3-cyclohexenyl)-butanol, Dihydroterpineol, 2,6-dimethylheptan-2-ol, 3-(4-methoxyphenyl)-2-methyl-Propanal, Octahydro-5-methoxy-4,7-Methano-1H-indene-2-carboxaldehyde, alpha-methyl-1,3-Benzodioxole-5-propanal, 3-[4-(1,1-dimethylethyl)phenyl]propanal, 8-(1-methylethyl)-1-oxaspiro[4.5]-decan-2-one, Tetrahydro-6-(2Z)-2-pentenyl-2H-Pyran-2-one, 5-(3Z)-3-hexenyldihydro-2(3H)-Furanone, 5-hexyldihydro-5-methyl-2(3H)-Furanone, 4-methyl-5-pentyl-dihydro-2(3H)-Furan-2-one, 6-hexyltetrahydro-2H-pyran-2-one, 5-hexyl-Furan-2(3H)-one, 6-heptyltetrahydro-2H-Pyran-2-one, 5-octyl-furan-2-one, Dihydro-5-pentyl-2(3H)-Furanone, 5-heptyldihydro-(2(3H)-Furanone), and 6-hexyltetrahydro-2H-Pyran-2-one.

5. The personal care product composition according to claim 1 further comprising at least one aromatic alcohol or derivative thereof selected from the group consisting of phenylethyl alcohol, phenyl propyl alcohol, benzyl formiate, and phenethyl formiate; and at least one antimicrobial fragrance ingredient selected from the group consisting of Geranium oil, Peppermint oil, Rose oil, Cinnamon leaf oil, Fucus oil, Clove bud oil, Clove leaf oil, Palmarosa oil, Citrus oil, Terpene fraction of citrus oil, Orange oil, Terpene fraction of orange oil, Geraniol, Cuminic alcohol, Perilla alcohol, Citronellol, Eugenol, Cinnamic alcohol, Nerol, Menthol, Borneol, Octan-1-ol, Nonan-1-ol, Decan-1-ol, Dec-9-en-1-ol, Limonene, 2,6-dimethyl-7-octen-2-ol, 3-methyl-5-phenyl-pentanol, 2-methyl-5-phenyl-pentanol, undec-10-en-1-ol, 4-(1-methylethyl)-cyclohexanol, 4-(1,1-dimethylethyl)-cyclohexanol, 2,2-dimethyl-3-(3-methyl phenyl)-propanol, 4-(1-methylethyl)cyclohexyl-methanol, (E)-2-(3,3-dimethylbicyclo[2.2.1]hept-2-ylidene)-ethanol, 3,7-dimethyl-7-octen-1-ol, 2-methoxy-4-propyl-phenol, 3-(4-methyl-3-cyclohexenyl)-butanol, Dihydroterpineol, 2,6-dimethylheptan-2-ol, 3-(4-methoxyphenyl)-2-methyl-Propanal, Octahydro-5-methoxy-4,7-Methano-1H-indene-2-carboxaldehyde, alpha-methyl-1,3-Benzodioxole-5-propanal, 3-[4-(1,1-dimethylethyl)-phenyl]propanal, 8-(1-methylethyl)-1-oxaspiro[4.5]-decan-2-one, Tetrahydro-6-(2Z)-2-pentenyl-2H-Pyran-2-one, 5-(3Z)-3-hexenyldihydro-2(3H)-Furanone, 5-hexyldihydro-5-methyl-2(3H)-Furanone, 4-methyl-5-pentyl-dihydro-2(3H)-Furan-2-one, 6-hexyltetrahydro-2H-pyran-2-one, 5-hexyl-Furan-2(3H)-one, 6-heptyltetrahydro-2H-Pyran-2-one, 5-octyl-furan-2-one, Dihydro-5-pentyl-2(3H)-Furanone, 5-heptyldihydro-(2(3H)-Furanone), and 6-hexyltetrahydro-2H-Pyran-2-one.

6. A composition according to claim 1 selected from compositions for personal care products applied to and left on the skin or scalp, optionally comprising at least one of creams, salves, lotions, or ointments for hand, face or body, perfumes, eau de Cologne, eau de toilet, deodorants, or antiperspirants; and products applied but rinsed off, optionally comprising at least one of soaps, liquid soaps, shower gels, or shampoos.

7. A personal care product comprising the composition of claim 1, in an application form selected from stick, roll-on, spray, pump-spray, aerosol, soap bar, powder, solution, gel, cream, balm and lotion.

8. A personal care product or composition therefor according to claim 1 wherein the personal care product composition comprises lipids.

9. A personal care product or composition therefor according to claim 8 wherein the composition is an emulsion.

10. A personal care product or a personal care product composition according to claim 1, wherein the pH is 5 to 9.

11. The personal care product composition according to claim 5, wherein the at least one benzaldehyde or benzaldehyde-derivative compound a) and the at least one fragrant acid b), the at least one aromatic alcohol or derivative thereof, and the at least one antimicrobial fragrance ingredient that are present in the composition exclusively are compounds or ingredients that occur in nature.

12. The personal care product composition according to claim 11 wherein all ingredients of the composition consists exclusively of compounds or ingredients that occur in nature.

13. The personal care product composition according to claim 11 wherein the compounds or ingredients that occur in nature have been extracted or purified from a natural source optionally comprising a botanical source, or have been formed by natural processes optionally comprising fermentation.

14. Method for the preparation of a preserved personal care product composition, or a preserved personal care product comprising admixing an effective amount of the at least one benzaldehyde or benzaldehyde-derivative compound a) and the at least one fragrant acid b) as defined in claim 1 to the cosmetically-acceptable base or a personal care product base.

15. Method of forming the personal care product composition as defined in claim 1 which is sufficiently bactericidal to have a reduction factor for Pseudomonas aeruginosa and Staphylococcus aureus of at least 1000 per 7 days, and which is sufficiently sporicidal to have a reduction factor of at least 100 per 7 days for Aspergillus niger, comprising
admixing an effective amount of the at least one benzaldehyde or benzaldehyde-derivative compound a) and an effective amount of the at least one fragrant acid b) to a personal care product base, wherein optionally said compounds a) and b) are added in a form of a pre-prepared fragrance composition comprising said compounds a) and b).

16. Method according to claim 15 wherein the total concentration of the at least one benzaldehyde or benzaldehyde-derivative compound a) in the personal care product composition is from 0.05% to 1% (w/w), optionally from 0.3 to 0.7%, and the total concentration of the at least one fragrant acid b) in the personal care product composition is from 0.05% to 1% (w/w), optionally 0.3 to 0.7%.

17. Method of claim 15 wherein the personal care product composition formed further comprises at least one aromatic alcohol or derivative thereof selected from the group consisting of phenylethyl alcohol, phenyl propyl alcohol, benzyl formiate, and phenethyl formiate.

18. Method of claim 15 wherein the personal care product composition formed further comprises at least one antimicrobial fragrance ingredient selected from the group consisting of Geranium oil, Peppermint oil, Rose oil, Cinnamon leaf oil, Fucus oil, Clove bud oil, Clove leaf oil, Palmarosa oil, Citrus oil, Terpene fraction of citrus oil, Orange oil, Terpene fraction of orange oil, Geraniol, Cuminic alcohol, Perilla alcohol, Citronellol, Eugenol, Cinnamic alcohol, Nerol, Menthol, Borneol, Octan-1-ol, Nonan-1-ol, Decan-1-ol, Dec-9-en-1-ol, Limonene, 2,6-dimethyl-7-octen-2-ol, 3-methyl-5-phenyl-pentanol, 2-methyl-5-phenyl-pentanol, undec-10-en-1-ol, 4-(1-methylethyl)-cyclohexanol, 4-(1,1-dimethylethyl)-cyclohexanol, 2,2-dimethyl-3-(3-methyl phenyl)-propanol, 4-(1-methylethyl)cyclohexyl-methanol, (E)-2-(3,3-dimethylbicyclo[2.2.1]hept-2-ylidene)-ethanol, 3,7-dimethyl-7-octen-1-ol, 2-methoxy-4-propyl-phenol, 3-(4-methyl-3-cyclohexenyl)-butanol, Dihydroterpineol, 2,6-dimethylheptan-2-ol, 3-(4-methoxyphenyl)-2-methyl-Propanal, Octahydro-5-methoxy-4,7-Methano-1H-indene-2-carboxaldehyde, alpha-methyl-1,3-Benzodioxole-5-propanal, 3-[4-(1,1-dimethylethyl)phenyl]propanal, 8-(1-methylethyl)-1-oxaspiro[4.5]-decan-2-one, Tetrahydro-6-(2Z)-2-pentenyl-2H-Pyran-2-one, 5-(3Z)-3-hexenyldihydro-2(3H)-Furanone, 5-hexyldihydro-5-methyl-2(3H)-Furanone, 4-methyl-5-pentyl-dihydro-2(3H)-Furan-2-one, 6-hexyltetrahydro-2H-pyran-2-one, 5-hexyl-Furan-2(3H)-one, 6-heptyltetrahydro-2H-Pyran-2-one, 5-octyl-furan-2-one, Dihydro-5-pentyl-2(3H)-Furanone, 5-heptyldihydro-(2(3H)-Furanone), and 6-hexyltetrahydro-2H-Pyran-2-one.

19. Method of claim 15 wherein the personal care product composition formed further comprises at least one aromatic alcohol or derivative thereof selected from the group consisting of phenylethyl alcohol, phenyl propyl alcohol, benzyl formiate, and phenethyl formiate; and at least one antimicrobial fragrance ingredient selected from the group consisting of Geranium oil, Peppermint oil, Rose oil, Cinnamon leaf oil, Fucus oil, Clove bud oil, Clove leaf oil, Palmarosa oil, Citrus oil, Terpene fraction of citrus oil, Orange oil, Terpene fraction of orange oil, Geraniol, Cuminic alcohol, Perilla alcohol, Citronellol, Eugenol, Cinnamic alcohol, Nerol, Menthol, Borneol, Octan-1-ol, Nonan-1-ol, Decan-1-ol, Dec-9-en-1-ol, Limonene, 2,6-dimethyl-7-octen-2-ol, 3-methyl-5-phenyl-pentanol, 2-methyl-5-phenyl-pentanol, undec-10-en-1-ol, 4-(1-methylethyl)-cyclohexanol, 4-(1,1-dimethylethyl)-cyclohexanol, 2,2-dimethyl-3-(3-methyl phenyl)-propanol, 4-(1-methylethyl)cyclohexyl-methanol, (E)-2-(3,3-dimethylbicyclo[2.2.1]hept-2-ylidene)-ethanol, 3,7-dimethyl-7-octen-1-ol, 2-methoxy-4-propyl-phenol, 3-(4-methyl-3-cyclohexenyl)-butanol, Dihydroterpineol, 2,6-dimethylheptan-2-ol, 3-(4-methoxyphenyl)-2-methyl-Propanal, Octahydro-5-methoxy-4,7-Methano-1H-indene-2-carboxaldehyde, alpha-methyl-1,3-Benzodioxole-5-propanal, 3-[4-(1,1-dimethylethyl)phenyl]propanal, 8-(1-methylethyl)-1-oxaspiro[4.5]-decan-2-one, Tetrahydro-6-(2Z)-2-pentenyl-2H-Pyran-2-one, 5-(3Z)-3-hexenyldihydro-2(3H)-Furanone, 5-hexyldihydro-5-methyl-2(3H)-Furanone, 4-methyl-5-pentyl-dihydro-2(3H)-Furan-2-one, 6-hexyltetrahydro-2H-pyran-2-one, 5-hexyl-Furan-2(3H)-one, 6-heptyltetrahydro-2H-Pyran-2-one, 5-octyl-furan-2-one, Dihydro-5-pentyl-2(3H)-Furanone, 5-heptyldihydro-(2(3H)-Furanone), and 6-hexyltetrahydro-2H-Pyran-2-one.

20. Method of claim 15 wherein the personal care product composition is or is brought into the form of a personal care product, optionally comprising at least one of sticks, roll-ons, sprays, pump-sprays, aerosols, soap bars, powders, solutions, gels, creams, balms, or lotions.
